(19) United States
(12) Patent Application Publication
(10) Pub. No.: US 2005/0234111 A1
(43) Pub. Date: Oct. 20, 2005

(54) D-homo-17-chlor-16(17)-ene steroids

(76) Inventors: Sven Ring, Jena (DE); Ralf Wyrwa, Rothenstein (DE); Guenter Kaufmann, Jena (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/101,655

(22) Filed: Apr. 8, 2005

Related U.S. Application Data
(60) Provisional application No. 60/560,353, filed on Apr. 8, 2004.

(51) Int. Cl. A61K 31/58; C07D 271/12; C07D 285/14

(52) U.S. Cl. 514/362; 514/379; 514/406; 548/126; 548/242; 548/358.5

(57) ABSTRACT

The invention relates to D-homo-17-chlor-16(17)-ene steroids of formula I with androgenic activity 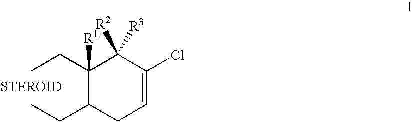 process for their production and pharmaceutical compositions that contain these compounds, as well as their use for the production of pharmaceutical agents.

This application claims the benefit of the filing date of U.S. Provisional Application Ser. No. 60/560,353 filed Apr. 8, 2004 which is incorporated by reference herein.

The invention relates to D-homo-17-chlor-16(17)-ene steroids of general formula I 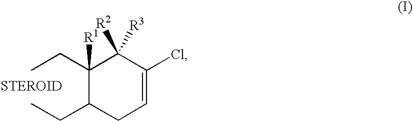
a process for their production, pharmaceutical compositions that contain these compounds as well as their use for the production of pharmaceutical agents with androgenic action.

WO 99/46279 discloses D-homo-16-chloro-17-oxosteroids that inhibit the 17β-hydroxy steroid-dehydrogenases. DE 19712488 discloses sulfamates of D-homostratrienes, which are unsubstituted at 17a-carbon, however. D-Homo-17a-chlor-16(17)-ene steroids show antiandrogenic action (EP 52799). D-Homo-17a-substituted-pregnanes- and androstanes, in which the 16(17)-double bond is lacking, are known from DE 2700267. Such compounds show anesthetic action.

Other D-homo-11β-aryl-17-chlor-16(17)-ene steroids that are strong antigestagens (DE 4042005, Steroids, 1994, 59, 176-80) are known from the prior art. Also known is 17aβ3-hydroxy-7α-methyl-D-homoestra-4,16-dien-3-one, which exhibits androgenic and antigonadotropic properties (Steroids, 1990, 55, 59-64).

D-Homo-17-chlor-16(17)-ene steroids of the androstane, 19-norandrostane and 13-ethylgonane series are not known to date.

The object of this invention therefore consists in preparing new compounds with androgenic action.

The object is achieved by D-homo-17-chlor-16(17)ene steroids of formula I 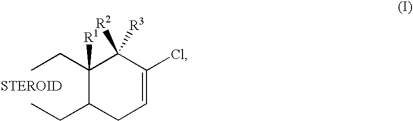
in which
R¹ stands for a C₁₋₆-alkyl group,
R² stands for a hydroxy group, a C₁₋₁₀-alkyloxy group, a C₁₋₁₅-acyloxy group, a C₆₋₁₅-cycloalkylacyloxy group, a C₇₋₁₅-arylacyloxy group, a C₇₋₁₅-arylalkyloxy group or a C₇₋₁₅-alkylaryloxy group, and
R³ stands for a hydrogen atom, a C₁₋₁₀-alkyl group, a C₁₋₁₀-perfluoroalkyl group,
   a radical —(CH₂)ₙCH₂W, with
      n=0, 1 or 2, and
      W stands for a hydroxy group, a halogen atom for a pseudohalogen or a C₁₋₁₀-alkyloxy group,
   a radical —(CH₂)ₘ—CH═CH(CH₂)ₚ—R⁴, with
      m=0, 1,2 or 3, p=0, 1 or 2, and
      R⁴ stands for a hydrogen atom, a C₁₋₁₀-alkyl radical, a C₆₋₁₅-aryl radical, a C₇₋₁₅-arylalkyl radical, a C₇₋₁₅-alkylaryl radical, a hydroxy group, a C₁₋₁₀-alkyloxy group or a C₁₋₁₀-acyloxy group, or
   a radical —(CH₂)ₒC≡CR⁵ with
      o=0, 1 or 2, and
      R⁵ stands for a hydrogen atom, a halogen atom, a C₁₋₁₀-alkyl radical, a C₆₋₁₅-aryl radical, a C₇₋₁₅-aralkyl radical, a C₇₋₁₅-alkylaryl radical or a C₁₋₁₀-acyloxy radical;
or
R² stands for a hydrogen atom, a C₁₋₁₀-alkyl group, a C₁₋₁₀-perfluoroalkyl group,
   a radical —(CH₂)ₙCH₂W, with
      n=0, 1 or 2, and
      W stands for a hydroxy group, a halogen atom for a pseudohalogen or a C₁₋₁₀-alkyloxy group,
   a radical —(CH₂)ₘ—CH═CH(CH₂)ₚ—R⁴ with
      m=0, 1, 2 or 3,
   p=0, 1 or 2 and
      R₄ stands for a hydrogen atom, a C₁₋₁₀-alkyl radical, a C₆₋₁₅-aryl radical, a C₇₋₁₅-arylalkyl radical, a C₇₋₁₅-alkylaryl radical, a hydroxy group, a C₁₋₁₀-alkyloxy group or a C₁₋₁₀-acyloxy group, or
   a radical —(CH₂)ₒC≡CR⁵ with
      o=0, 1 or 2 and R⁵ stands for a hydrogen atom, a halogen atom, a C₁₋₁₀-alkyl radical, a C₆₋₁₅-aryl radical, a C₇₋₁₅-aralkyl radical, a C₇₋₁₅-alkylaryl radical or a C₁₋₁₀-acyloxy radical;
         and
R³ stands for a hydroxy group, a C₁₋₁₀-alkyloxy group, a C₁₋₁₅-acyloxy group, a C₆₋₁₅-cycloalkylacyloxy group, a C₇₋₁₅-arylacyloxy group, a C₇₋₁₅-arylalkyloxy group or a C₇₋₁₅-alkylaryloxy group,
or
R² and R³ together stand for a keto group, a methylene group or a difluoromethylene group, or with the inclusion of the 17a-C atom, R² and R³ form a spirooxirane or a 2,2-dimethyl-1,3-dioxolane,
and
STEROID stands for a steroidal partial ring system of the formulas A, B, C, D, E and F, which are presented below, 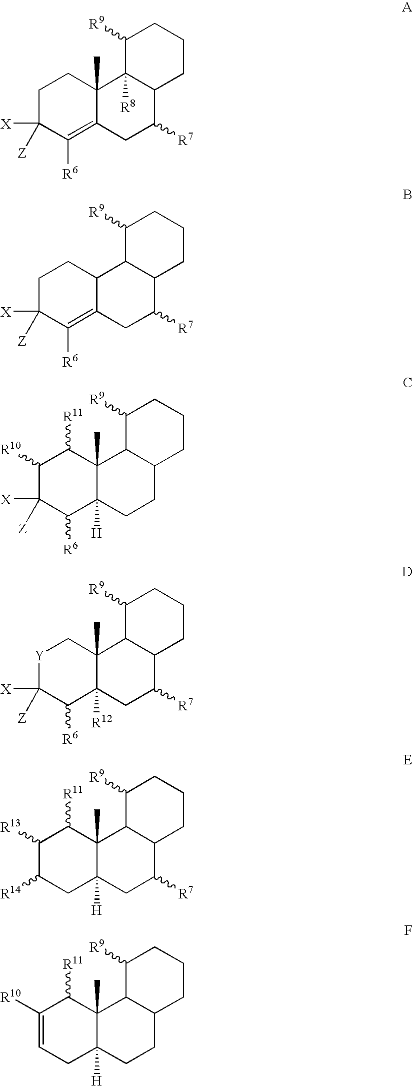
   whereby
in A, an additional double bond can be found in 1,2-position, and
in B, one or two additional double bonds can be found in 9,10-position and 11,12-position,
   in which
R⁶ means a hydrogen atom, a halogen atom, a hydroxy group, a methyl group or a trifluoromethyl group,
X and Z in each case mean a hydrogen atom or together an oxygen atom or a hydroxyimino group,
R⁷ means a hydrogen atom, a C₁₋₆ alkyl group or a C₁₋₆ alkenyl group,
R⁸ means a hydrogen atom, a halogen atom, and together with R⁹ a double bond,
R⁹ means a hydrogen atom, a hydroxy group, a halogen atom, a methyl group or an ethyl group or together with R⁸ a double bond,
R¹⁰ means a hydrogen atom, a methyl group, a nitrile group, a hydroxymethylene group or a formyl group,
R¹¹ means a hydrogen atom, a methyl group, or a nitrile group,
R¹⁰ and R¹¹, in addition to the above-mentioned meanings, together mean a double bond or a methylene bridge,
R¹² means a hydrogen atom or together with R⁶ a double bond,
R¹³ and R¹⁴ together mean a double bond, an oxirane ring, a thiirane ring, a [2,3c] oxadiazole ring, a [3,2c]isoxazole ring or a [3,2c] pyrazole ring, and Y means an oxygen or nitrogen atom,
   and their pharmaceutically acceptable salts.

The wavy lines at R⁶, R⁷, R⁸, R¹¹, R¹², R¹³, and R¹⁴ mean that these substituents can be in α- or β-position.

The compounds according to the invention have androgenic activity.

The C₁₋₆-alkyl group is a branched or unbranched alkyl radical, which is formed by, for example, a methyl group, an ethyl group, an n-propyl group, an i-propyl group, an n-butyl group, an i-butyl group or a tert.-butyl group, an n-pentyl group, an i-pentyl group, an n-hexyl group, a 2-methylpentyl group, a 3-methylpentyl group, a 2,2-dimethylbutyl group, or a 2,3-dimethylbutyl group.

The term halogen atom stands for a fluorine atom, a chlorine atom, a bromine atom or an iodine atom.
R¹ preferably means a methyl group or an ethyl group, whereby the methyl group is especially preferred.
R² preferably means a hydroxy group or an esterified hydroxy group, in particular a formyloxy group, an acetyloxy group, a propanoyloxy group, a butyryloxy group, a [(trans-4-butylcyclohexyl)carbonyl]oxy group, a phenylpropanoyloxy group, an iso-butyryloxy group or an undecanoyloxy group.
R³ preferably means a hydrogen atom, or a methyl group, an ethyl group, an ethinyl group, a hydroxymethyl group, a chloromethyl group, a bromomethyl group, a cyanomethyl group, an azidomethyl group, a rhodanomethyl group, or a methoxymethyl group.

Substituents for R² and R³ that are indicated above are also preferred in the exchange of substituent R² for R³, whereby the above-mentioned variant is especially preferred.
R⁶ preferably means a hydrogen atom, an F atom, a Cl atom, a Br atom, a hydroxy group, a methyl group or a trifluoromethyl group,
X and Z together preferably mean an oxygen atom,
R⁷ preferably means a hydrogen atom or a methyl group,
R⁸ preferably means a hydrogen atom or a fluorine atom,
R⁹ preferably means a hydrogen atom, a hydroxy group, a methyl group, a fluorine atom or a chlorine atom,
R¹⁰ preferably means a hydrogen atom, a methyl group, a formyl group or a nitrile group,
R¹¹ preferably means a hydrogen atom or a methyl group,
R¹² preferably means a hydrogen atom, a hydroxymethyl group or a formyl group,
R¹³ and R¹⁴ together preferably mean a thiirane ring, a [2,3c] oxadiazole ring, a [3,2c] isoxazole ring or a [3,2c] pyrazole ring, and
Y preferably means an oxygen atom.

If STEROID stands for a steroidal ring system of partial formula A,
R¹ preferably means a methyl group,
R⁶ preferably means a fluorine atom, a chlorine atom, a bromine atom, a trifluoromethyl group or a hydroxy group,
R⁷ preferably means a methyl group,
R⁸ preferably means a fluorine atom and/or
R⁹ preferably means a hydroxy group.

If STEROID stands for a steroidal ring system of partial formula B,
R¹ preferably means a methyl group,
R⁶ preferably means a fluorine atom, a chlorine atom, a bromine atom, a trifluoromethyl group or a hydroxy group,
R⁷ preferably means a methyl group and/or
R⁹ preferably means a hydroxy group.

If STEROID stands for a ring system of partial formula C,
R⁶ preferably means a fluorine atom, a chlorine atom, a bromine atom, a trifluoromethyl group or a hydroxy group,
R⁷ preferably means a methyl group,
R⁹ preferably means a hydroxy group and/or
R¹² preferably means a hydroxymethyl group or a formyl group.

If STEROID stands for a ring system of partial formula D,
R¹ means a methyl group,
R⁶ means a fluorine atom, a chlorine atom, a bromine atom, a trifluoromethyl group or a hydroxy group,
R⁷ means a methyl group and/or
Y means an oxygen atom.

If STEROID stands for a ring system of partial formula E,
R¹ preferably means a methyl group and/or
R⁹ preferably means a hydroxy group.

Especially preferred compounds are cited below:
1) 17-Chloro-17aβ-hydroxy-17a-homoandrosta-4,16-dien-3-one (1),
2) 17-Chloro-17aβ-hydroxy-7α-methyl-17a-homoandrosta-4,16-dien-3-one (2),
3) 4,17-Dichloro-17aβ-hydroxy-7α-methyl-17a-homoandrosta-4,16-dien-3-one,
4) 17-Chloro-4,17aβ-dihydroxy-7α-methyl-17a-homoandrosta-4,16-dien-3-one,
5) 17-Chloro-4,17aβ-dihydroxy-17a-homoandrosta-4,16-dien-3-one (4),
6) 4,17-Dichloro-17aβ-hydroxy-17a-homoandrosta-4,16-dien-3-one, (3),
7) 117-Chloro-17aβ-hydroxy-4-bromo-17a-homoandrosta-4,16-dien-3-one,
8) 17-Chloro-17aβ-hydroxy-4-fluoro-17a-homoandrosta-4,16-dien-3-one,
9) 17-Chloro-17aβ-hydroxy-4-trifluoromethyl-17a-homoandrosta-4,16-dien-3-one,
10) 17-Chloro-11β,17aβ-dihydroxy-17a-homoandrosta-4,16-dien-3-one,
11) 17-Chloro-11β,17aβ-dihydroxy-9α-fluoro-17a-homoandrosta-4,16-dien-3-one,
12) 17-Chloro 17aβ-hydroxy-17a-homoandrosta-1,4,16-trien-3-one (5),
13) 4,17-Dichloro-17aβ-hydroxy-17a-homoandrosta-1,4,16-trien-3-one (6),
14) 17-Chloro-4,17aβ-dihydroxy-17a-homoandrosta-1,4,16-trien-3-one,
15) 17-Chloro-17aβ-hydroxy-70α-methyl-17a-homoandrosta-1,4,16-trien-3-one,
16) 4,17-Dichloro-17aβ-hydroxy-7α-methyl-17a-homoandrosta-1,4,16-trien-3-one,
17) 17-Chloro-17aβ-hydroxy-17a-homo-estra-4,16-dien-3-one (7),
18) 17-Chloro-17aβ-hydroxy-7α-methyl-17a-homo-estra-4,16-dien-3-one (8),
19) 4,17-Dichloro-17aβ-hydroxy-7α-methyl-17a-homo-estra-4,16-dien-3-one,
20) 17-Chloro-4,17aβ-dihydroxy-7α-methyl-17a-homo-estra-4,16-dien-3-one,
21) 17-Chloro-4,17aβ-dihydroxy-17a-homo-estra-4,16-dien-3-one (10),
22) 4,17-Dichloro-17aβ-hydroxy-17a-homo-estra-4,16-dien-3-one (9),
23) 17-Chloro-17aβ-hydroxy-4-bromo-17a-homo-estra-4,16-dien-3-one,
24) 17-Chloro-17aβ-hydroxy-4-fluoro-17a-homo-estra-4,16-dien-3-one,
25) 17-Chloro-17aβ-hydroxy-4-trifluoromethyl-17a-homo-estra-4,16-dien-3-one,
26) 17-Chloro-11β,17aβ-dihydroxy-17a-homo-estra-4,16-dien-3-one,
27) 17-Chloro-11β,17aβ-methyl-17a-homo-estra-4,16-dien-3-one,
28) 17-Chloro-17aβ-hydroxy-7α,11β-dimethyl-17a-homo-estra-4,16-dien-3-one,
29) 4,17-Dichloro-17aβ-hydroxy-17a-homo-estra-4,9,16-trien-3-one,
30) 17-Chloro-17aβ-hydroxy-17a-homo-estra-4,9,11,16-tetraen-3-one,
31) 17-Chloro-17aβ-hydroxy-7α-methyl-17a-homo-estra-4,9,11,16-tetraen-3-one,
32) 4,17-Dichloro-17aβ-hydroxy-7α-methyl-17a-homo-estra-4,9,11,16-tetraen-3-one,
33) 17-Chloro-17aβ-hydroxy-13-ethyl-17a-homo-estra-4,16-dien-3-one,
34) 17-Chloro-17aβ-hydroxy-7α-methyl-13-ethyl-17a-homo-estra-4,16-dien-3-one,
35) 4,17-Dichloro-17aβ-hydroxy-7α-methyl-13-ethyl-17a-homo-estra-4,16-dien-3-one,
36) 17-Chloro-4,17aβ-dihydroxy-7α-methyl-13-ethyl-17a-homo-estra-4,16-dien-3-one,
37) 17-Chloro-4,17aβ-dihydroxy-13-ethyl-17a-homo-estra-4,16-dien-3-one,
38) 4,17-Dichloro-17aβ-hydroxy-13-ethyl-17a-homo-estra-4,16-dien-3-one,
39) 17-Chloro-17aβ-hydroxy-4-bromo-13-ethyl-17a-homo-estra-4,16-dien-3-one,
40) 17-Chloro-17aβ-hydroxy-4-fluoro-13-ethyl-17a-homo-estra-4,16-dien-3-one,
41) 17-Chloro-17aβ-hydroxy-4-trifluoromethyl-13-ethyl-17a-homo-estra-4,16-dien-3-one,
42) 17-Chloro-11β,17aβ-dihydroxy-13-ethyl-17a-homo-estra-4,16-dien-3-one,
43) 17-Chloro-11β,17aβ-dimethyl-13-ethyl-17a-homo-estra-4,16-dien-3-one,
44) 17-Chloro-17aβ-hydroxy-7α,11β-dimethyl-13-ethyl-17a-homo-estra-4,16-dien-3-one,
45) 4,17-Dichloro-17aβ-hydroxy-13-ethyl-17a-homo-estra-4,9,16-trien-3-one,
46) 17-Chloro-17aβ-hydroxy-13-ethyl-17a-homo-estra-4,9,11,16-tetraen-3-one,
47) 17-Chloro-17aβ-hydroxy-7α-methyl-13-ethyl-17a-homo-estra-4,9,11,16-tetraen-3-one,
48) 4,17-Dichloro-17aβ-hydroxy-7α-methyl-13-ethyl-17a-homo-estra-4,9,11,16-tetraen-3-one,
49) 17-Chloro-17aβ-hydroxy-17a-homo-5α-androst-16-en-3-one (11),
50) 17-Chloro-17aβ-hydroxy-17a-homo-7α-methyl -5α-androst-16-en-3-one,
51) 17-Chloro-17aβ-hydroxy-17a-homo-2-hydroxymethylene-5α-androst-16-en-3-one (13),
52) 17-Chloro-17aβ-hydroxy-17a-homo-2α-methyl-5α-androst-16-en-3-one,
53) 17-Chloro-17aβ-hydroxy-17a-homo-1α-methyl-5α-androst-16-en-3-one,
54) 17-Chloro-17aβ-hydroxy-17a-homo-5α-androsta-2,16-diene,
55) 17-Chloro-17aβ-hydroxy-17a-homo-2-methyl-5α-androsta-2,16-diene,
56) 17-Chloro-17aβ-hydroxy-17a-homo-2-cyano-5α-androsta-2,16-diene,
57) 17-Chloro-17aβ-hydroxy-17a-homo-2-formyl-5α-androsta-2,16-diene,
58) 17-Chloro-17aβ-hydroxy-17a-homo-[2,3c]oxadiazole-5α-androst-16-ene,
59) 17-Chloro-17aβ-hydroxy-17a-homo-[3,2c]isoxazole-5α-androst-16-ene,
60) 17-Chloro-17aβ-hydroxy-17a-homo-[3,2c]pyrazole-5α-androst-16-ene,
61) 17-Chloro-17aβ-hydroxy-17a-homo-2β,3β-epithio-5α-androst-16-ene,
62) 17-Chloro-17aβ-hydroxy-17a-homo-2α,3α-epithio-5α-androst-16-ene,
63) 17-Chloro-17aβ-hydroxy-17a-homo-2-oxa-5α-androst-16-en-3-one (14),
64) 17-Chloro-17aβ-hydroxy-17a-homo-5α-androsta-1,16-dien-3-one (12),
65) 17-Chloro-17aβ-hydroxy-17a-homo-1-methyl-5α-androsta-1,16-dien-en-3-one,
66) 17-Chloro-17aβ-hydroxy-17a-homo-2-methyl-5α-androsta-1,16-dien-en-3-one,
67) 17-Chloro-17aα-methyl-17aβ-hydroxy-17a-homoandrosta-4,16-dien-3-one,
68) 17-Chloro-17aα-methyl-17aβ-hydroxy-7α-methyl-17a-homoandrosta-4,16-dien-3-one,
69) 17-Chloro-17aα-ethyl-17aβ-hydroxy-17a-homoandrosta-4,16-dien-3-one,
70) 17-Chloro-17aα-ethyl-17aβ-hydroxy-7α-methyl-17a-homoandrosta-4,16-dien-3-one,
71) 17-Chloro-17aα-ethinyl-17aβ-hydroxy-17a-homoandrosta-4,16-dien-3-one,
72) 17-Chloro-17aα-ethinyl-17aβ-hydroxy-7α-methyl-17a-homoandrosta-4,16-dien-3-one,
73) 17-Chloro-17aα-hydroxymethyl-17aβ-hydroxy-17a-homoandrosta-4,16-dien-3-one,
74) 17-Chloro-17aα-hydroxymethyl-17aβ-hydroxy-7α-methyl-17a-homoandrosta-4,16-dien-3-one,
75) 17-Chloro-17aα-chloromethyl-17aβ-hydroxy-17a-homoandrosta-4,16-dien-3-one,
76) 17-Chloro-17aα-chloromethyl-17aβ-hydroxy-7α-methyl-17a-homoandrosta-4,16-dien-3-one,
77) 17-Chloro-17aα-bromomethyl-17aβ-hydroxy-17a-homoandrosta-4,16-dien-3-one,
78) 17-Chloro-17aα-bromomethyl-17aβ-hydroxy-7α-methyl-17a-homoandrosta-4,16-dien-3-one,
79) 17-Chloro-17aα-cyanomethyl-17aβ-hydroxy-17a-homoandrosta-4,16-dien-3-one,
80) 17-Chloro-17aα-cyanomethyl-17aβ-hydroxy-7α-methyl-17a-homoandrosta-4,16-dien-3-one,
81) 17-Chloro-17aα-azidomethyl-17aβ3-hydroxy-17a-homoandrosta-4,16-dien-3-one,
82) 17-Chloro-17aα-azidomethy-17aβ-hydroxy-7α-methyl-17a-homoandrosta-4,16-dien-3-one,
83) 17-Chloro-17aα-rhodanomethyl-17aβ-hydroxy-17a-homoandrosta-4,16-dien-3-one,
84) 17-Chloro-17aα-rhodanomethyl-17aβ-hydroxy-7α-methyl-17a-homoandrosta-4,16-dien-3-one,
85) 17-Chloro-17aα-methoxymethyl-17aβ3-hydroxy-17a-homoandrosta-4,16-dien-3-one,
86) 17-Chloro-17aα-methoxymethyl-17aβ-hydroxy-7α-methyl-17a-homoandrosta-4,16-dien-3-one,
87) 17-Chloro-17aα-methyl-17aβ-hydroxy-17a-homo-estra-4,16-dien-3-one,
88) 17-Chloro-17aα-methyl-17aβ-hydroxy-7α-methyl-17a-homo-estra-4,16-dien-3-one,
89) 17-Chloro-17aα-ethyl-17aβ-hydroxy-17a-homo-estra-4,16-dien-3-one,
90) 17-Chloro-17aα-ethyl-17aβ-hydroxy-7α-methyl-17a-homo-estra-4,16-dien-3-one,
91) 17-Chloro-17aα-ethinyl-17aβ-hydroxy-17a-homo-estra-4,16-dien-3-one,
92) 17-Chloro-17aα-ethinyl-17aβ-hydroxy-7α-methyl-17a-homo-estra-4,16-dien-3-one,
93) 17-Chloro-17aα-hydroxymethyl-17aβ-hydroxy-17a-homo-estra-4,16-dien-3-one,
94) 17-Chloro-17aα-hydroxymethyl-17aβ-hydroxy-7α-methyl-17a-homo-estra-4,16-dien-3-one,
95) 17-Chloro-17aα-chloromethyl-17aβ-hydroxy-17a-homo-estra-4,16-dien-3-one,
96) 17-Chloro-17aα-chloromethy-17aβ-hydroxy-7α-methyl-17a-homo-estra-4,16-dien-3-one,
97) 17-Chloro-17aα-bromomethyl-17aβ-hydroxy-17a-homo-estra-4,16-dien-3-one,
98) 17-Chloro-17aα-bromomethyl-17aβ-hydroxy-7α-methyl-17a-homo-estra-4,16-dien-3-one,
99) 17-Chloro-17aα-cyanomethyl-17aβ-hydroxy-17a-homo-estra-4,16-dien-3-one,
100) 17-Chloro-17aα-cyanomethyl-17aβ-hydroxy-7α-methyl-17a-homo-estra-4,16-dien-3-one,
101) 17-Chloro-17aα-azidomethyl-17aβ-hydroxy-1,7a-homo-estra-4,16-dien-3-one,
102) 17-Chloro-17aα-azidomethyl-17aβ-hydroxy-7α-methyl-17a-homo-estra-4,16-dien-3-one,
103) 17-Chloro-17aα-rhodanomethyl-17aβ-hydroxy-17a-homo-estra-4,16-dien-3-one,
104) 17-Chloro-17aα-rhodanomethyl-17aβ-hydroxy-7α-methyl-17a-homo-estra-4,16-dien-3-one,
105) 17-Chloro-17aα-methoxymethyl-17aβ-hydroxy-17a-homo-estra-4,16-dien-3-one,
106) 17-Chloro-17aα-methoxymethyl-17aβ-hydroxy-7α-methyl-17a-homo-estra-4,16-dien-3-one.

The compounds according to the invention have androgenic activity in vitro and also in vivo (Hershberger test on rats).

The receptor binding affinities for the androgen receptor that are cited below illustrate the in vitro activity.

Receptor Binding Tests

Measurement of the androgen receptor binding affinity:

The receptor binding affinity was determined by competitive binding of a specifically binding 3H-labeled hormone (tracer) and the compound to be tested on receptors in cystosol from animal target organs. In this case, receptor saturation and reaction equilibrium were desired.

The tracer and increasing concentrations of the compound to be tested (competitor) were co-incubated at 0-4° C. over 18 hours with the receptor-containing cytosol fraction. After separation of the unbonded tracer with carbon-dextran suspension, the receptor-bonded tracer portion was measured for each concentration and the IC50 was determined from the concentration series. The relative molar binding affinity (RBA) was calculated (RBA of reference substance=100%) as a quotient of the IC50 values of the reference substance and the compound to be tested (×100%).

The following incubation conditions were selected:

Prostate cytosol of the castrated rat; prostates stored at −30° C.;

| | |
|---|---|
| Buffer: | TED with 10% glycerol as well as 2 |
| | μmol of triamcinolone acetonide. |
| Tracer: | 3H-Metribolone 4 nmol; |
| Reference substance: | 5α-Dihydrotestosterone (DHT). |

| Compound | RBA % |
|---|---|
| DHT | 100 |
| 2 | 46 |
| 9 | 51 |
| 17 | 80 |
| 18 | 105 |
| 21 | 70 |

The D-homo-17chlor-16(17)-ene steroids according to the invention advantageously show a very high binding the androgen receptor.

These test results open up many possibilities in the compounds of general formula (I) according to the invention for birth control in men and women, hormone replacement therapy (HRT) in men and women, or the treatment of hormonally-induced diseases in men and women, such as, for example, endometriosis, breast cancer or hypogonadism.

Subjects of this invention are therefore also pharmaceutical compositions that contain at least one D-homo-17-chlor-16(17)-ene steroid of general formula (I) or its salt, optionally together with pharmaceutically compatible adjuvants and vehicles.

These pharmaceutical compositions and pharmaceutical agents can be provided for oral, rectal, vaginal, subctaneous, percutaneous, transdermal, buccal, intravenous or intrasmuscular administration. In addition to commonly used vehicles and/or diluents, they contain at least one compound of general formula I or salt thereof.

The pharmaceutical agents according to this invention are produced in a known way with commonly used solid or liquid vehicles or diluents and the commonly used pharmaceutical-technical adjuvants corresponding to the desired type of administration—with a suitable dosage. The preferred preparations consist in a dispensing form that is suitable for oral administration. Such dispensing forms are, for example, tablets, film tablets, coated tablets, capsules, pills, patches, powders, solutions or suspensions or else depot forms.

Of course, parenteral preparations, such as injection solutions, are also considered. In addition, for example, suppositories and agents for vaginal administration can also be mentioned as preparations.

Corresponding tablets can be obtained by, for example, mixing the active ingredient with known adjuvants, for example inert diluents such as dextrose, sugar, sorbitol, mannitol, polyvinylpyrrolidone, explosives such as corn starch or alginic acid, binders such as starch or gelatin, lubricants such as magnesium stearate or talc, and/or agents for achieving a depot effect. such as carboxylpolymethylene, carboxylmethyl cellulose, cellulose acetate phthalate or polyvinyl acetate. The tablets can also consist of several layers.

Coated tablets can accordingly be produced by coating cores, which are produced analogously to the tablets, with agents that are commonly used in tablet coatings, for example polyvinylpyrrolidone or shellac, gum arabic, talc, titanium oxide or sugar. In this case, the coated tablet shell can also consist of several layers, and the adjuvants that are mentioned above in the tablets can be used.

Solutions or suspensions with the compounds of general formula I according to the invention can contain additional taste-enhancing agents such as saccharine, cyclamate or sugar, as well as, e.g., aromatic substances such as vanilla or orange extract. In addition, they can contain suspending adjuvants such as sodium carboxymethyl cellulose or preservatives such as p-hydroxybenzoates.

The capsules that contain compounds of general formula I can be produced by, for example, the compound(s) of general formula I being mixed with an inert vehicle such as lactose or sorbitol and encapsulated in gelatin capsules.

Suitable suppositories can be produced by, for example, mixing with vehicles that are provided for this purpose, such as neutral fats or polyethylene glycol or derivatives thereof.

For the production of compounds of general formula I with partial structures A-F, known steroid bases can be used. The following steroid bases can be used, for example:

| | |
|---|---|
| For A: | 6-Methoxy-3,5-cyclo-androstan-17-one |
| For B: | 3,3-Dimethoxy-estr-5(10)-en-17-one |
| | (DD 79-213049), 18ahomo-3,3- |
| | dimethoxy-estr-5(10)-en-17-one |

For C,D,E,F: Epiandrosterone
   or compounds of general formula II are used with partial structures A-F that are protected in a suitable way according to the methods that are known to one skilled in the art.

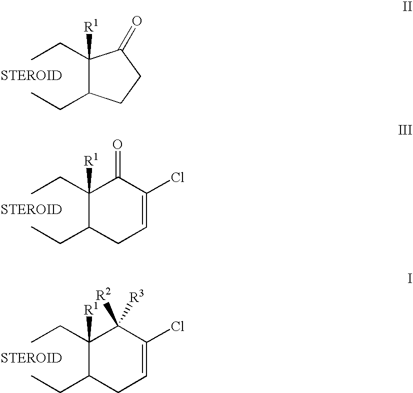

For the production of the compounds of general formula I with partial structures A-F, enol compounds of the 17-ketones of general formula II with partial structures A-F, which are protected in a suitable way, or the above-mentioned steroid bases are reacted with dichlorocarbene, whereby compounds of general formula III are obtained in which STEROID can mean partial structures A-F or the steroid bases. As enol compounds of 17-ketones, preferably trialkylsilylenol ether can be used. As known to one skilled in the art, dichlorocarbene can be obtained from chloroform by treatment with bases, such as, for example, potassium hydroxide, or from the sodium salt of trichloroacetic acid by heating in a suitable solvent, such as, for example, dimethoxyethane, tetrachloroethylene or chloroform, advantageously with the addition of a phase transfer catalyst. The compounds of general formula III are then reduced according to methods that are generally familiar to one skilled in the art and optionally are substituted to obtain compounds of general formula I.

For the formation of pharmaceutically compatible salts of the compounds of general formula I according to the invention, i.a., hydrochloric acid, hydrobromic acid, sulfuric acid and phosphoric acid are considered as inorganic acids, and, i.a., acetic acid, propionic acid, maleic acid, fumaric acid, succinic acid, benzoic acid, ascorbic acid, oxalic acid, salicylic acid, tartaric acid, citric acid, lactic acid, malic acid, mandelic acid, cinnamic acid and methanesulfonic acid are considered as organic acids.

Below, the invention is explained based on the examples.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever. In the foregoing and in the following examples, all temperatures are set forth uncorrected in degrees Celsius and, all parts and percentages are by weight, unless otherwise indicated.

### EXAMPLE 1

### Synthesis of 17-Chloro-17aβ-hydroxy-17a-homoandrosta-4,16-dien-3-one

### Stage 1 6-Methoxy-3,5-cyclo-androstan-17-one

35 g of dehydroepiandrosterone is dissolved in 150 ml of pyridine and mixed at 0° C. with 58 g of toluenesulfonic acid chloride. After 24 hours, it is poured into ice water and acidified with concentrated hydrochloric acid. It is suctioned off and washed with water. The residue is refluxed in 11 of methanol with 20 g of sodium acetate for 1 hour. It is concentrated by evaporation to 200 ml and worked up in extract form with chloroform. 6-Methoxy-3,5-cyclo-androstan-17-one is obtained as a yellow oil.

### Stage 2 6-Methoxy-3,5-cyclo-17-[(trimethylsilyl)oxy]-androstan-16-ene

37 g of 6-methoxy-3,5-cyclo-androstan-17-one is dissolved in 150 ml of THF and mixed at −60° C. with 100 ml of 2 M lithium diisopropylamide solution. After 1 hour, 27 ml of trimethylchlorosilane is added, and it is allowed to heat to room temperature. It is poured into 11 of saturated sodium bicarbonate solution and worked up in extract form with ethyl acetate. The 6-methoxy-3,5-cyclo-17-[(trimethylsilyl)oxy]-androstan-16-ene that is obtained is directly further processed.

### Stage 3 17-Chloro-6-methoxy-3,5-cyclo-17a-homo-androstan-16-en-17a-one

6-Methoxy-3,5-cyclo-17-[(trimethylsilyl)oxy]-androstan-16-ene is refluxed in 400 ml of chloroform with 141 g of trichloroacetic acid sodium salt and 3.3 g of benzyltriethylammonium chloride for 2 hours. It is poured into 11 of saturated sodium bicarbonate solution, worked up in extract form with chloroform, and the residue is chromatographed on silica gel. 17-Chloro-6-methoxy-3,5-cyclo-17a-homo-androstan-16-en-17a-one is obtained.

¹H-NMR (CDCl₃): 1.02 (s, 3H, H-18), 1.10 (s, 3H, H-19), 3.35 (s, 3H, 6-OMe), 7.02 (dd, 1H, J=2.3, 6.2 Hz, H-16)

### Stage 4 17-Chloro-6-methoxy-3,5-cyclo-17a-homo-androstan-16-en-17aβ-ol

24 g of 17-chloro-6-methoxy-3,5-cyclo-17a-homoandrostan-16-en-17a-one is reduced in 400 ml of methanol with 8.5 g of sodium borohydride with the addition of 17 g of cerium(Ill) nitrate at 0° C. Then, it is concentrated by evaporation to half the volume, acidified with 30% acetic acid and worked up in extract form by means of chloroform. The 17-chloro-6-methoxy-3,5-cyclo-17a-homoandrostan-16-en-17aβ-ol that is obtained is directly further processed.

### Stage 5 17aβ-Acetoxy-17-chloro-6-methoxy-3,5-cyclo-17a-homo-androstan-16-ene

23 g of 17-chloro-6-methoxy-3,5-cyclo-17a-homoandrostan-16-en-17aβ-ol is acetylated in 60 ml of pyridine and 50 ml of acetic acid anhydride. After working-up, 17aβ-acetoxy-17-chloro-6-methoxy-3,5-cyclo-17a-homo-androstan-16-ene, which is directly further processed, is obtained.

### Stage 6 17aβ-Acetoxy-17-chloro-17a-homo-androsta-5,16-dien-313-ol

25 g of 17aβ-acetoxy-17-chloro-6-methoxy-3,5-cyclo-17a-homoandrostan-16-ene is refluxed in 200 ml of acetone, 21 ml of water and 6 ml of 60% perchloric acid for 1 hour. Then, it is neutralized with 10% sodium bicabonate solution and concentrated by evaporation. The residue is worked up in extract form with ethyl acetate. After the organic extract is concentrated by evaporation, 17aβ-acetoxy-17-chloro-17a-homoandrosta-5,16-dien-3β-ol, which is directly further processed, is obtained.

### Stage 7 17aβ-Acetoxy-17-chloro-17a-homoandrosta-4,16-dien-3-one

24 g of 17aβ-acetoxy-17-chloro-17a-homoandrosta-5,16-dien-313-ol is refluxed in 350 ml of toluene with 174 ml of cyclohexanone and 10 g of aluminum isopropylate for 1 hour. After working-up in extract form and after chromatography on silica gel, 17aβ-acetoxy-17-chloro-17a-homoandrosta-4,16-dien-3-one, which is directly further processed, is obtained.

### Stage 8 17-Chloro-17aβ-hydroxy-17a-homoandrosta-4,16-dien-3-one

12 g of 17aβ3-acetoxy-17-chloro-17a-homoandrosta-4,16-dien-3-one is refluxed in a solution of 8 g of potassium hydroxide in 200 ml of methanol for 1 hour. The solution is acidified with 1N hydrochloric acid and almost completely concentrated by evaporation. The residue is worked up in extract form with water and ethyl acetate. After the organic extract is concentrated by evaporation, 17-chloro-17aβ-hydroxy-17a-homoandrosta-4,16-dien-3-one is obtained.

¹H-NMR(CDCl₃): 0.91(s, 3H, H-18), 1.18 (s, 3H, H-19), 3.80 (m, 1H, H-17a), 5.73 (s, 1H, H-4), 5.87 (dd, 1H, J=1.9, 5.4Hz, H-16)

### EXAMPLE 2

### Synthesis of 17-Chloro-17aβ-hydroxy-7α-methyl-17a-homoandrosta-4,16-dien-3-one

### Stage 1 17-Chloro-17aβ-hydroxy-17a-homoandrosta-4,6,16-trien-3-one:

5 g of 17-chloro-17aβ-hydroxy-17a-homoandrosta-4,16-dien-3-one is refluxed with 7 g of chloranil in 200 ml of tert.-butanol for 30 minutes. It is allowed to cool and evaporated to the dry state. The residue is chromatographed on silica gel. 17-Chloro-17aβ-hydroxy-17a-homoandrosta-4,6,16-trien-3-one is obtained.

### Stage 2 17-Chloro-17aβ-hydroxy-7α-methyl-17a-homoandrosta-4,16-dien-3-one

80 ml of THF is added to a solution of methylmagesium iodide (prepared from 2.5 g of magnesium and 6.4 ml of methyl iodide in 80 ml of diethyl ether), cooled to −5° C., and 1 g of copper acetate-monohydrate (dissolved in 50 ml of THF) is added. It is cooled to −20° C., and then a solution of 5 g of 17-chloro-17aβ-hydroxy-17a-homoandrosta-4,6,16-trien-3-one in 80 ml THF is added in drops. After 2 hours, it is poured into ice water/1 M sulfuric acid and extracted with 3×80 ml of ethyl acetate. The organic extract is dried and concentrated by evaporation. The residue is chromatographed on silica gel. For further purification, it is recrystallized from ethyl acetate. 17-Chloro-17aβ-hydroxy-7α-methyl-17a-homoandrosta-4,16-dien-3-one is obtained.

¹H-NMR (CDCl₃): 0.75 (d, 3H, J=7 Hz, 7-Me), 0.91 (s, 3H, H-18), 1.18 (s, 3H, H-19), 3.81 (m, 1H, H-17a), 5.73 (s, 1H, H-4), 5.86 (m, 1H, H-16)

### EXAMPLE 3

### Synthesis of 4,17-Dichloro-17aβ-hydroxy-17a-homoandrosta-4,16-dien-3-one

### Stage 1: 17-Chloro-17aβ-hydroxy-4ξ,5ξ-Epoxy 17a-homoandrost-16-en-3-one

2 g of 17-chloro-17aβ-hydroxy-17a-homoandrosta-4,16-dien-3-one is dissolved in 120 ml of methanol as well as 70 ml of THF and mixed at 10° C. with 20 ml of hydrogen peroxide solution (35%). While being stirred, 5 ml of 10% sodium hydroxide solution is added, and it is stirred for 3 hours. The reaction solution is concentrated by evaporation to 50 ml, then mixed with 50 ml of dichloromethane and 25 ml of water, and the organic phase is separated. It is washed with semiconcentrated thiosulfate solution, dried and evaporated to the dry state. The residue that is obtained consists of a mixture of 4α,5α- or 4β,5β-epoxides and is used without further purification in the next stage.

### Stage 2 4,17-Dichloro-17aβ-hydroxy-17a-homoandrosta-4,16-dien-3-one

2 g of epoxide mixture from Stage 1 is dissolved in 200 ml of acetone and mixed at 5° C. with 12 ml of concentrated hydrochloric acid. After 2 hours, it is neutralized with soda solution, and the acetone is drawn off. The residue is extracted out with dichloromethane. The organic extracts are dried and concentrated by evaporation. After crystallization from ethyl acetate, 4,17-dichloro-17aβ-hydroxy-17a-homoandrosta-4,16-dien-3-one is obtained.

¹H-NMR (CDCl₃): 0.91 (s, 3H, H-18), 1.23 (s, 3H, H-19), 3.81 (m, 1H, H-17a), 5.87 (m, 1H, H-16)

### EXAMPLE 4

### Synthesis of 17-Chloro-4,17aβ-dihydroxy-17a-homoandrosta-4,16-dien-3-one

2 g of the epoxide mixture of 17-chloro-17aβ-hydroxy-4ξ,5ξ-epoxy-17a-homoandrost-16-en-3-one is dissolved in 20 ml of acetic acid, which contains 2 vol % of concentrated sulfuric acid. The solution is allowed to stand for 24 hours at 10° C. Then, it is mixed with 200 ml of ethyl acetate and neutralized with soda solution. The organic phase is dried and concentrated by evaporation. The residue is chromatographed on silica gel and crystallized from ethyl acetate. ¹H-NMR (CDCl₃): 0.91 (s, 3H, H-18), 1.16 (s, 3H, H-19), 3.81 (m, 1H, H-17a), 5.87 (m, 1H, H-16), 6.08 (s, 1H, 4-OH).

### EXAMPLE 5

### Synthesis of 17-Chloro-17aβ-hydroxy-17a-homoandrosta-1,4,16-trien-3-one

2 g of 17-chloro-17aβ-hydroxy-17a-homoandrosta-4,16-dien-3-one is stirred with 1.8 g of DDQ in 60 ml of toluene for 60 hours at 85° C. Precipitate is filtered out, rewashed with toluene, and the filtrate is concentrated by evaporation. The residue is chromatographed on silica gel and recrystallized from ethyl acetate.

¹H-NMR (CDCl₃): 0.95 (s, 3H, H-18), 1.23 (s, 3H, H-19), 3.79.(sbr, 1H, H-17a), 5.85 (m, 1H, H-16), 6.07 (m, 1H, H-4), 6.24 (dd, J=1.9, 10 Hz, 1H, H-2), 7.07 (d, J=10 Hz, 1H, H-1)

### EXAMPLE 6

### Synthesis of 4,17-Dichloro-17aβ-hydroxy-17a-homoandrosta-1,4,16-trien-3-one

Production analogous to instructions for 17-chloro-17aβ-hydroxy-17a-homoandrosta-1,4,16-trien-3-one from 4,17-dichloro-17a13-hydroxy-17a-homoandrosta-4,16-dien-3-one.

### EXAMPLE 7

### Synthesis of 17-Chloro-17aβ-hydroxy-17a-homo-estra-4,16-dien-3-one

### Stage 1 3,3-Dimethoxy-17-[(trimethylsilyl)oxy]-estra-5(10),16-diene

37 g of 3,3-dimethoxy-estr-5(10)-en-17-one is dissolved in 150 ml of THF and mixed at −60° C. with 100 ml of 2 M lithium diisopropylamide solution. After 1 hour, 27 ml of trimethylchlorosilane is added, and it is allowed to heat to room temperature. It is poured into 11 of saturated sodium bicarbonate solution and worked up in extract form with ethyl acetate. The 3,3-dimethoxy-17-[(trimethylsilyl)oxy]-estra-5(10),16-diene that is obtained is directly further processed.

### Stage 2 17-Chloro-17a-homo-3,3-dimethoxy-estra-5(10),16-dien-17a-one

3,3-Dimethoxy-17-[(trimethylsilyl)oxy]-estra-5(10),16-diene is refluxed in 400 ml of chloroform with 141 g of trichloroacetic acid sodium salt and 3.3 g of benzyltriethylammonium chloride for 2 hours. It is poured into 11 of saturated sodium bicarbonate solution, worked up in extract form with chloroform, and the residue is chromatographed on silica gel for purification. 17-Chloro-17a-homo-3,3-dimethoxy-estra-5(10),16-dien-17a-one is obtained.

¹H-NMR (CDCl₃): 1.06 (s, 3H, H-18), 3.21 (s, 3H, OMe), 3.24 (s, 3H, OMe), 7.02 (m, 1H, H-16)

### Stage 3 17-Chloro-17aβ-hydroxy-17a-homo-estra-4,16-dien-3-one

24 g of 17-chloro-17a-homo-3,3-dimethoxy-estra-5(10),16-dien-17a-one is reduced in 400 ml of methanol with 8.5 g of sodium borohydride with the addition of 17 g of cerium(III) nitrate at 0° C. Then, it is concentrated by evaporation to half the volume, acidified with 30% acetic acid and worked up in extract form by means of chloroform. The residue is stirred in 400 ml of methanol with the addition of 50 ml of 6N sulfuric acid for 2 hours at 45-50° C. After neutralization with saturated sodium bicarbonate solution, 300 ml is distilled off in a vacuum, and it is worked up in extract form. For purification, it is chromatographed on silica gel, and 17-chloro-17aβ-hydroxy-17a-homo-estra-4,16-dien-3-one is obtained.

¹H-NMR (CDCl₃): 0.92 (s, 3H, H-18), 3.82 (sbr, 1H, H-17a), 5.83 (m, 1H, H-4), 5.87 (m, 1H, H-16)

### EXAMPLE 8

### Synthesis of 17-Chloro-17aβ-hydroxy-7α-methyl-177a-homo-estra-4,16-dien-3-one

### Stage 1 17-Chloro-17aβ-hydroxy-17a-homo-estra-4,6,16-trien-3-one

17-Chloro-17aβ-hydroxy-17a-homo-estra-4,6,16-trien-3-one can easily be obtained from 17-chloro-17a-hydroxy-17a-homo-estra-4,16-dien-3-one according to the method of J. A. Campbell et al (Steroids 1963, 317).

### Stage 2 17-Chloro-17aβ-hydroxy-7α-methyl-17a-homo-estra-4,16-dien-3-one

80 ml of THF is added to a solution of methylmagnesium iodide (prepared from 2.5 g of magnesium and 6.4 ml of methyl iodide in 80 ml of diethyl ether), it is cooled to −5° C., and 1 g of copper acetate-monohydrate (dissolved in 50 ml of THF) is added. It is cooled to −20° C., and then a solution of 5 g of 17-chloro-17αβ-hydroxy-17a-homo-estra-4,6,16-trien-3-one in 80 ml of THF is added in drops. After 2 hours, it is poured into ice water/2N sulfuric acid and extracted with 3×80 ml of ethyl acetate. The organic extract is dried and concentrated by evaporation. The residue is chromatographed on silica gel. For further purification, it is recrystallized from ethyl acetate. 17-Chloro-17aβ-hydroxy-7α-methyl-17a-homo-estra-4,16-dien-3-one is obtained.

¹H-NMR (CDCl₃): 0.75 (d, 3H, J=7 Hz, 7-Me), 0.93 (s, 3H, H-18), 3.83 (m, 1H, H-17a), 5.83 (s, 1H, H-4), 5.87 (m, 1H, H-16)

### EXAMPLE 9

### Synthesis of 4,17-Dichloro-17aβ-hydroxy-17a-homo-estra-4,16-dien-3-one

### Stage 1 17-Chloro-17aβ-hydroxy-4ξ,5ξ-Epoxy 17a-homo-estra-16-en-3-one

2 g of 17-chloro-17aβ-hydroxy-17a-homo-estra-4,16-dien-3-one is dissolved in 120 ml of methanol as well as 70 ml of THF and mixed at 10° C. with 20 ml of hydrogen peroxide solution (35%). While being stirred, 5 ml of 10% sodium hydroxide solution is added, and it is stirred for 3 hours. The reaction solution is concentrated by evaporation to 50 ml, then mixed with 50 ml of dichloromethane and 25 ml of water, and the organic phase is separated. It is washed with semiconcentrated thiosulfate solution, dried and evaporated to the dry state. The residue that is obtained consists of a mixture of 4α,5α- or 4β,5β-epoxides and is used without further purification in the next stage.

### Stage 2 4,17-Dichloro-17a-hydroxy-17a-homo-estra-4,16-dien-3-one

2 g of epoxide mixture (Stage 1) is dissolved in 200 ml of acetone and mixed at 5° C. with 12 ml of concentrated hydrochloric acid. After 2 hours, it is neutralized with soda solution, and the acetone is drawn off. The residue is extracted out with dichloromethane. The organic extracts are dried and concentrated by evaporation. After crystallization from ethyl acetate, 4,17-dichloro-17aβ-hydroxy-17a-homo-estra-4,16-dien-3-one is obtained.

¹H-NMR (CDCl₃): 0.94 (s, 3H, H-18), 3.83 (m, 1H, H-17a), 5.88 (m, 1H, H-16).

### EXAMPLE 10

### Synthesis of 17-Chloro-4,17aβ-dihydroxy-17a-homo-estra-4,16-dien-3-one

2 g of the epoxide mixture of 17-chloro-17aβ-hydroxy-4ξ,5ξ-epoxyl 7a-homo-estra-16-en-3-one is dissolved in 20 ml of acetic acid, which contains 2 vol % of concentrated sulfuric acid. The solution is allowed to stand for 24 hours at 10° C. Then, it is mixed with 200 ml of ethyl acetate and neutralized with soda solution. The organic phase is dried and concentrated by evaporation. The residue is chromatographed on silica gel and crystallized from ethyl acetate.

¹H-NMR (CDCl₃): 0.92 (s, 3H, H-18), 3.82 (m, 1H, H-17a), 5.87 (m, 1H, H-16), 6.10 (s, 1H, 4-OH)

### EXAMPLE 11

### Synthesis of 17-Chloro-17aβ-hydroxy-17a-homo-5α-H-androst-16-en-3-one

### Stage 1 3β-Trimethylsilyloxy-5α-H-androstan-17-one

10 g of epiandrosterone is dissolved in 75 ml of DMF and 60 ml of pyridine and mixed at room temperature with 25 ml of trimethylchlorosilane. After 90 minutes, it is poured into 300 ml of saturated sodium bicarbonate solution, suctioned off, washed with water and drawn in a dry state. 3β-Trimethylsilyloxy-5α-H-androstan-17-one is obtained.

### Stage 2 3,17-Di-(trimethylsilyloxy)-5α-H-androst-16-ene

12 g of 3β-trimethylsilyloxy-5α-H-androstan-17-one is dissolved in 50 ml of THF and mixed at −60° C. with 33 ml of 2 M lithium diisopropylamide solution. After 1 hour, 9 ml of trimethylchlorosilane is added, and it is allowed to heat to room temperature. It is poured into 300 ml of saturated sodium bicarbonate solution and worked up in extract form with ethyl acetate. The 3,17-di-(trimethylsilyloxy)-5α-H-androst-16-ene that is obtained is directly further processed.

### Stage 3 17-Chloro-3β-Trimethylsilyloxy-17a-homo-5α-H-androst-16-en-17-one

3β,17-Di-(trimethylsilyloxy)-5α-H-androst-16-ene is refluxed in 400 ml of chloroform with 47 g of trichloroacetic acid sodium salt and 1.1 g of benzyltriethylammonium chloride for 2 hours. It is poured into 300 ml of saturated sodium bicarbonate solution, worked up in extract form with chloroform, and the residue is chromatographed on silica gel for purification. 17-Chloro-3β-trimethylsilyloxy-17a-homo-5α-H-androst-16-en-17-one is obtained.

### Stage 4 17-Chloro-3β-acetoxy-17a-homo-5α-H-androst-16-en-17aβ-ol

8 g of 17-chloro-3β-trimethylsilyloxy-17a-homo-5α-H-androst-16-en-17-one is reacted in 200 ml of THF with 8 g of tetrabutylammonium fluoride. After working-up in extract form with ethyl acetate, 17-chloro-3β-hydroxy-17a-homo-5α-H-androst-16-en-17-one, which is acetylated [with] 20 ml of pyridine and 15 ml of acetic acid anhydride, is obtained. The thus obtained 17-chloro-3β-acetoxy-17a-homo-5α-H-androst-16-en-17-one is reduced in 200 ml of methanol with 4 g of sodium borohydride with the addition of 6 g of cerium(III) nitrate at 0° C. Then, it is concentrated by evaporation to half the volume, acidified with 30% acetic acid, and worked up in extract form by means of chloroform. 17-Chloro-3β-acetoxy-17a-homo-5α-H-androst-16-en-17aβ-ol is obtained:

¹H-NMR (CDCl₃): 0.80 (s, 3H, H-18), 0.85 (s, 3H, H-19), 2.02 (s, 3H, 3-acetate), 3.79 (m, 1H, H-17a), 4.68 (m, 1H, 3-H), 5.85 (m, 1H, H-16):

### Stage 5 17-Chloro-17aβ-tetrahydropyranyloxy-17a-homo-5α-H-androst-16-en-3-one

The 17-chloro-3β-acetoxy-17a-homo-5α-H-androst-16-en-17aβ-ol (5 g) that is obtained is converted with 3,4-dihydro-2H-pyran in the presence of pyridinium tosylate in 100 ml of dichloromethane into the 17aβ-tetrahydropyranyl ether, and the acetate in 3β-position is saponified with 5 g of potassium hydroxide in 100 ml of boiling methanol. 5 g of 17-chloro-17aβ-tetrahydropyranyloxy-17a-homo-5α-H-androst-16-en-3β-ol, which is refluxed in 120 ml of toluene with 35 ml of cyclohexanone and 2 g of aluminum isopropylate for 1 hour, is obtained. After working-up in extract form and after chromatographyon silica gel, 17-chloro-17aβ-tetrahydropyranyloxy-17a-homo-5α-H-androst-16-en-3-one is obtained.

### Stage 6 17-Chloro-17aβ-hydroxy-17a-homo-5α-H-androst-16-en-3-one

3 g of 17-chloro-17aβ-tetrahydropyranyloxy-17a-homo-5α-H-androst-16-en-3-one is stirred in 150 ml of 80% acetic acid for 2 hours at 60° C. It is neutralized with 2N sodium hydroxide solution and worked up in extract form. After chromatographic purification on silica gel, 17-chloro-17aβ-hydroxy-17a-homo-5α-H-androst-16-en-3-one is obtained:

¹H-NMR (CDCl₃): 0.88 (s, 3H, H-18), 1.00 (s, 3H, H-19), 3.80 (m, 1H, H-17a), 5.86 (m, 1H, H-16).

### EXAMPLE 12

### Synthesis of 17-Chloro-17aβ-hydroxy-17a-homo-5α-H-androsta-1,16-dien-3-one

5 g of 17-chloro-17aβ-hydroxy-17a-homo-5α-H-androst-16-en-3-one is mixed with 5 g of pyridinium hydrobromide-perbromide while being stirred in 100 ml of THF. After 15 minutes, 250 ml of saturated sodium bicarbonate solution is added, extracted with chloroform, dried, and concentrated by evaporation. The residue is refluxed with 5 g of lithium carbonate and 10 g of lithium bromide in 100 ml of DMF for 6 hours. It is allowed to cool, diluted with 500 ml of toluene, washed with water, dried and concentrated by evaporation. For purification, it is chromatographed on silica gel and recrystallized from ethyl acetate. 17-Chloro-17aβ-hydroxy-17a-homo-5α-H-androsta-1,16-dien-3-one is obtained:

¹H-NMR (CDCl3): 0.91 (s, 3H, H-18), 0.99 (s, 3H, H-19), 3.82 (m, 1H, H-17a), 5.85 (m, 1H, H-2), 5.86 (m, 1H, H-16), 7.12 (d, J=10 Hz, 1H, H-1).

### EXAMPLE 13

### Synthesis of 17-Chloro-17aβ-hydroxy-17a-homo-2-hydroxymethylene-5α-H-androst-16-en-3-one

4 g of 17-chloro-17aβ-hydroxy-17a-homo-5α-H-androst-16-en-3-one is mixed in 150 ml of toluene with 3.2 g of sodium hydride and 8 ml of formic acid ethyl ester. After 24 hours, it is carefully hydrolyzed with water. It is acidified with 5N hydrochloric acid, the organic phase is separated, dried and concentrated by evaporation. For purification, it is chromatographed on silica gel and crystallized from acetone/hexane. 17-Chloro-17aβ-hydroxy-2-hydroxymethylene-17a-homo-5α-H-androst-16-en-3-one is obtained: ¹H-NMR (DMSO-d6): 0.65 (s, 3H, H-18), 0.74 (s, 3H, H-19), 5.19 (d, 1H, J=7 Hz, 2-CHO), 5.79 (m, 1H, H-16).

### EXAMPLE 14

### Synthesis of 17-Chloro-17aβ-hydroxy-17a-homo-2-oxa-5α-H-androst-16-en-3-one

4 g of 17-Chloro-17aβ-hydroxy-17a-homo-5α-H-androsta-1,16-dien-3-one is reacted in 200 ml of 90% acetic acid with 30 g of lead tetraacetate and 280 mg of osmium tetroxide. After 24 hours at room temperature, it is diluted with 500 ml of water and extracted three times with chloroform. The combined organic phases are alkalized with 2N sodium hydroxide solution and extracted three times with 200 ml of 2N sodium hydroxide solution. The combined aqueous phases are acidified with 5N hydrochloric acid and extracted three times with chloroform. The combined organic phases are dried and concentrated by evaporation. The residue is dissolved in 80 ml of THF and 80 ml of methanol. While being stirred, a solution of 1 g of sodium bicarbonate in 75 ml of water and 4.2 g of sodium borohydride are added in succession. After 2 hours, it is acidified with concentrated hydrochloric acid, extracted with ethyl acetate, dried and concentrated by evaporation. For purification, it is chromatographed on silica gel and recrystallized from ethyl acetate. 17-Chloro-17aβ-hydroxy-2-oxa-17a-homo-5α-H-androst-16-en-3-one is obtained:

¹H-NMR (CDCl3): 0.88 (s, 3H, H-18), 0.98 (s, 3H, H-19), 2.22 (dd, J=19.1, 13.1 Hz 1H, H-4), 2.53 (dd, J=18.7, 5.8 Hz, 1H, H-4), 3.80 (m, 1H, H-17a), 3.94 (d, J=10 Hz; 1H, H-1), 4.26 (d, J=10 Hz, 1H, H-1), 5.85 (m, 1H, H-16).

The entire disclosures of all applications, patents and publications, cited herein and of corresponding U.S. Provisional Application Ser. No. 60/560,353, filed Apr. 8, 2004, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. D-Homo-17-chlor-16(17)ene steroids of formula I 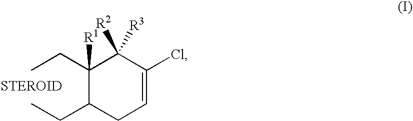
in which
R¹ stands for a C₁₋₆-alkyl group,
R² stands for a hydroxy group, a C₁₋₁₀-alkyloxy group, a C₁₋₁₅-acyloxy group, a C₆₋₁₅-cycloalkylacyloxy group, a C₇₋₁₅-arylacyloxy group, a C₇₋₁₅-arylalkyloxy group or a C₇₋₁₅-alkylaryloxy group, and
R³ stands for a hydrogen atom, a C₁₋₁₀-alkyl group, a C₁₋₁₀-perfluoroalkyl group,
a radical —(CH₂)ₙCH₂W, with
n=0, 1 or 2, and
W stands for a hydroxy group, a halogen atom for a pseudohalogen or a C₁₋₁₀-alkyloxy group,
a radical —(CH₂)ₘ—CH═CH(CH₂)ₚ—R⁴, with
m=0, 1, 2 or 3, p=0, 1 or 2, and
R⁴ stands for a hydrogen atom, a C₁₋₁₀-alkyl radical, a C₆₋₁₅-aryl radical, a C₇₋₁₅-arylalkyl radical, a C₇₋₁₅-alkylaryl radical, a hydroxy group, a C₁₋₁₀-alkyloxy group or a C₁₋₁₀-acyloxy group, or
a radical —(CH₂)ₒC≡CR⁵ with
o=0, 1 or 2, and
R⁵ stands for a hydrogen atom, a halogen atom, a C₁₋₁₀-alkyl radical, a C₆₋₁₅-aryl radical, a C₇₋₁₅-aralkyl radical, a C₇₋₁₅-alkylaryl radical or a C₁₋₁₀-acyloxy radical;
or
R² stands for a hydrogen atom, a C₁₋₁₀-alkyl group, a C₁₋₁₀-perfluoroalkyl group,
a radical —(CH₂)ₙCH₂W, with
n=0, 1 or 2, and
W stands for a hydroxy group, a halogen atom for a pseudohalogen or a C₁₋₁₀-alkyloxy group,
a radical —(CH₂)ₘ—CH═CH(CH₂)ₚ—R⁴ with
m=0, 1, 2 or 3,
p=0, 1 or 2 and
R⁴ stands for a hydrogen atom, a C₁₋₁₀-alkyl radical, a C₆₋₁₅-aryl radical, a C₇₋₁₅-arylalkyl radical, a C₇₋₁₅-alkylaryl radical, a hydroxy group, a C₁₋₁₀-alkyloxy group or a C₁₋₁₀-acyloxy group, or
a radical —(CH₂)ₒC≡CR⁵ with
o=0, 1 or 2 and R⁵ stands for a hydrogen atom, a halogen atom, a C₁₋₁₀-alkyl radical, a C₆₋₁₅-aryl radical, a C₇₋₁₅-aralkyl radical, a C₇₋₁₅-alkylaryl radical or a C₁₋₁₀-acyloxy radical;
and
R³ stands for a hydroxy group, a C₁₋₁₀-alkyloxy group, a C₁₋₁₅-acyloxy group, a C₆₋₁₅-cycloalkylacyloxy group, a C₇₋₁₅-arylacyloxy group, a C₇₋₁₅-arylalkyloxy group or a C₇₋₁₅-alkylaryloxy group,
or
R² and R³ together stand for a keto group, a methylene group or a difluoromethylene group, or with the inclusion of the 17a-C atom, R² and R³ form a spirooxirane or a 2,2-dimethyl-1,3-dioxolane,
and
STEROID stands for a steroidal partial ring system of the formulas A, B, C, D, E and F, which are presented below, 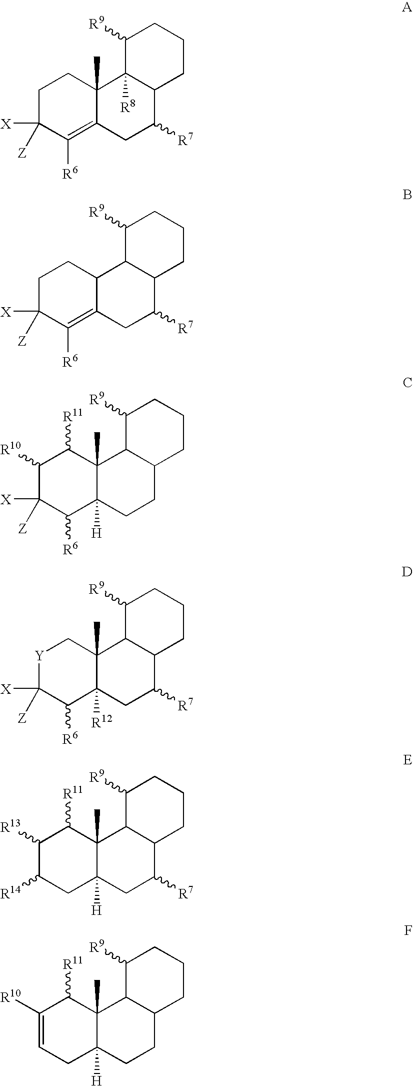
whereby
in A, an additional double bond can be found in 1,2-position, and
in B, one or two additional double bonds can be found in 9,10-position and 11,12-position,
in which
R⁶ means a hydrogen atom, a halogen atom, a hydroxy group, a methyl group or a trifluoromethyl group,
X and Z in each case mean a hydrogen atom or together an oxygen atom or a hydroxyimino group,
R⁷ means a hydrogen atom, a C₁₋₆ alkyl group or a C₁₋₆ alkenyl group,
R⁸ means a hydrogen atom, a halogen atom, and together with R⁹ a double bond,
R⁹ means a hydrogen atom, a hydroxy group, a halogen atom, a methyl group or an ethyl group or together with R⁸ a double bond,
R¹⁰ means a hydrogen atom, a methyl group, a nitrile group, a hydroxymethylene group or a formyl group,
R¹¹ means a hydrogen atom, a methyl group, or a nitrile group,
R¹⁰ and R¹¹, in addition to the above-mentioned meanings, together mean a double bond or a methylene bridge,
R¹² means a hydrogen atom or together with R⁶ a double bond,
R¹³ and R¹⁴ together mean a double bond, an oxirane ring, a thiirane ring, a [2,3c]oxadiazole ring, a [3,2c]isoxazole ring or a [3,2c]pyrazole ring, and Y means an oxygen or nitrogen atom,
and their pharmaceutically acceptable salts.

2. Compounds according to claim 1, characterized in that R¹ stands for a methyl group or an ethyl group.

3. Compounds according to claim 1, wherein R² or R³ represents a hydroxy group or an esterified hydroxy group, in particular a formyloxy group, an acetyloxy group, a propanoyloxy group, a butyryloxy group, a [(trans-4-butylcyclohexyl)-carbony]oxy group, a phenylpropanoyloxy group, an iso-butyryloxy group or an undecanoyloxy group

4. Compounds according to claim 1, wherein R³ or R² means a hydrogen atom, a methyl group, an ethyl group, an ethinyl group, a propinyl group, a hydroxymethyl group, a chloromethyl group, a bromomethyl group, a cyanomethyl group, an azidomethyl group, a rhodanomethyl group, or a methoxymethyl group.

5. Compounds according to claim 3, wherein one of the two substituents R² or R³ means a hydrogen atom.

6. Compounds according to claim 1, wherein R⁶ represents a hydrogen atom, an F atom, a Cl atom, or a Br atom, a hydroxy group, a methyl group or a trifluoromethyl group.

7. Compounds according to claim 1, wherein X and Z together represent an oxygen atom.

8. Compounds according to claim 1, wherein R⁷ represents a hydrogen atom or a methyl group.

9. Compounds according to claim 1, wherein R⁸ represents a hydrogen atom or a fluorine atom.

10. Compounds according to claim 1, wherein R⁹ represents a hydrogen atom, a hydroxy group, a methyl group, a fluorine atom or a chlorine atom.

11. Compounds according to claim 1, wherein R¹⁰ represents a hydrogen atom, a methyl group, a formyl group or a nitrile group.

12. Compounds according to claim 1, wherein R¹¹ represents a hydrogen atom or a methyl group.

13. Compounds according to claim 1, wherein R¹² represents a hydrogen atom, a hydroxymethyl group or a formyl group.

14. Compounds according to claim 1, wherein R¹³ and R¹⁴ together represent a thliirane ring, a [2,3c]oxadiazole ring, a [3,2c]isoxazole ring or a [3,2c]pyrazole ring.

15. Compounds according to claim 1, wherein Y represents an oxygen atom.

16. Compounds according to claim 1, wherein STEROID stands for a steroidal ring system of partial formula A, whereby the compound exhibits at least one of the features mentioned below: R¹ stands for a methyl group, R⁶ stands for a fluorine atom, for a chlorine atom, for a bromine atom, for a hydroxy group or a trifluoromethyl group, R⁷ stands for a methyl group, for a fluorine atom, and R⁹ stands for a hydroxy group.

17. Compounds according to claim 1, wherein STEROID stands for a steroidal ring system of partial formula B, whereby the compound exhibits at least one of the features mentioned below: R¹ stands for a methyl group, R⁶ stands for a fluorine atom, a chlorine atom, a bromine atom, a hydroxy group, or a trifluoromethyl group, R⁷ stands for a methyl group, and R⁹ stands for a hydroxy group.

18. Compounds according to claim 1, wherein STEROID stands for a steroidal ring system of partial formula C, whereby the compound exhibits at least one of the features mentioned below: R⁶ stands for a fluorine atom, a chlorine atom, a bromine atom, a hydroxy group or a trifluoromethyl group, R⁷ stands for a methyl group, R⁹ stands for a hydroxy group, and R¹⁰ stands for a hydroxymethyl group or a formyl group.

19. Compounds according to claim 1, wherein STEROID stands for a steroidal ring system of partial formula D, whereby the compound exhibits at least one of the features mentioned below: R¹ stands for a methyl group, R⁶ stands for a fluorine atom, a chlorine atom, a bromine atom, a hydroxy group or a trifluoromethyl group, R⁷ stands for a methyl group, R⁹ stands for a hydroxy group, and y stands for an oxygen atom.

20. Compounds according to claim 1, wherein STEROID stands for a steroidal ring system of partial formula E, whereby the compound exhibits at least one of the features mentioned below: R¹ stands for a methyl group, and R⁹ stands for a hydroxy group.

21. Compounds according to claim 1, namely:
1) 17-Chloro-17aβ-hydroxy-17a-homoandrosta-4,16-di en-3-one (1),
2) 17-Chloro-17aβ-hydroxy-7α-methyl-17a-homoandrosta-4,16-dien-3-one (2),
3) 4,17-Dichloro-17aβ-hydroxy-7α-methyl-17a-homoandrosta-4,16-dien-3-one,
4) 17-Chloro-4,17aβ-dihydroxy-7α-methyl-17a-homoandrosta-4,16-dien-3-one,
5) 17-Chloro-4,17aβ-dihydroxy-17a-homoandrosta-4,16-dien-3-one (4),
6) 4,17-Dichloro-17aβ-hydroxy-17a-homoandrosta-4,16-dien-3-one, (3),
7) 17-Chloro-17aβ-hydroxy-4-bromo-17a-homoandrosta-4,16-dien-3-one,
8) 17-Chloro-17aβ-hydroxy-4-fluoro-17a-homoandrosta-4,16-dien-3-one,
9) 17-Chloro-17aβ-hydroxy-4-trifluoromethyl-17a-homoandrosta-4,16-dien-3-one,
10) 17-Chloro-11β,17aβ-dihydroxy-17a-homoandrosta-4,16-dien-3-one,
11) 17-Chloro-11β,17aβ-dihydroxy-9α-fluoro-17a-homoandrosta-4,16-dien-3-one,
12) 17-Chloro-17aβ-hydroxy-17a-homoandiosta-1,4,16-trien-3-one (5),
13) 4,17-Dichloro-17aβ-hydroxy-17a-homoandrosta-1,4,16-trien-3-one (6),
14) 17-Chloro-4,17aβ-dihydroxy-17a-homoandrosta-1,4,16-trien-3-one,
15) 17-Chloro-17aβ-hydroxy-7α-methyl-17a-homoandrosta-1,4,16-trien-3-one,
16) 4,17-Dichloro-17aβ-hydroxy-7α-methyl-17a-homoandrosta-1,4,16-trien-3-one,
17) 17-Chloro-17aβ-hydroxy-17a-homo-estra-4,16-dien-3-one (7),
18) 17-Chloro-17aβ-hydroxy-7α-methyl-17a-homo-estra-4,16-dien-3-one (8),
19) 4,17-Dichloro-17aβ-hydroxy-7α-methyl-17a-homo-estra-4,16-dien-3-one,
20) 17-Chloro-4,17aβ-dihydroxy-7α-methyl-17a-homo-estra-4,16-dien-3-one,
21) 17-Chloro-4,17aβ-dihydroxy-17a-homo-estra-4,16-dien-3-one (10),
22) 4,17-Dichloro-17aβ-hydroxy-17a-homo-estra-4,16-dien-3-one (9),
23) 17-Chloro-17aβ-hydroxy-4-bromo-17a-homo-estra-4,16-dien-3-one,
24) 17-Chloro-17aβ-hydroxy-4-fluoro-17a-homo-estra-4,16-dien-3-one,
25) 17-Chloro-17aβ-hydroxy-4-trifluoromethyl-17a-homo-estra-4,16-dien-3-one,
26) 17-Chloro-11β,17aβ-dihydroxy-17a-homo-estra-4,16-dien-3-one,
27) 17-Chloro-11β,17aβ-dimethyl-17a-homo-estra-4,16-dien-3-one,
28) 17-Chloro-17aβ-hydroxy-7α,11β-dimethyl-17a-homo-estra-4,16-dien-3-one,
29) 4,17-Dichloro-17aβ-hydroxy-17a-homo-estra-4,9,16-trien-3-one,
30) 17-Chloro-17aβ-hydroxy-17a-homo-estra-4,9,11,16-tetraen-3-one,
31) 17-Chloro-17aβ-hydroxy-7α-methyl-17a-homo-estra-4,9,11,16-tetraen-3-one,
32) 4,17-Dichloro-17aβ-hydroxy-7α-methyl-17a-homo-estra-4,9,11,16-tetraen-3-one,
33) 17-Chloro-17aβ-hydroxy-13-ethyl-17a-homo-estra-4,16-dien-3-one,
34) 17-Chloro-17aβ-hydroxy-7α-methyl-13-ethyl-17a-homo-estra-4,16-dien-3-one,
35) 4,17-Dichloro-17aβ-hydroxy-7α-methyl-13-ethyl-17a-homo-estra-4,16-dien-3-one,
36) 17-Chloro-4,17aβ-dihydroxy-7α-methyl-13-ethyl-17a-homo-estra-4,16-dien-3-one,
37) 17-Chloro-4,17aβ-dihydroxy-13-ethyl-17a-homo-estra-4,16-dien-3-one,
38) 4,17-Dichloro-17aβ-hydroxy-13-ethyl-17a-homo-estra-4,16-dien-3-one,
39) 17-Chloro-17aβ-hydroxy-4-bromo-13-ethyl-17a-homo-estra-4,16-dien-3-one,
40) 17-Chloro-17aβ-hydroxy-4-fluoro-13-ethyl-17a-homo-estra-4,16-dien-3-one,
41) 17-Chloro-17aβ-hydroxy-4-trifluoromethyl-13-ethyl-17a-homo-estra-4,16-dien-3-one,
42) 17-Chloro-11β,17aβ-dihydroxy-13-ethyl-17a-homo-estra-4,16-dien-3-one,
43) 17-Chloro-11β,17aβ-dimethyl-13-ethyl-17a-homo-estra-4,16-dien-3-one,
44) 17-Chloro-17aβ-hydroxy-7α,11β-dimethyl-13-ethyl-17a-homo-estra-4,16-dien-3-one,
45) 4,17-Dichloro-17aβ-hydroxy-13-ethyl-17a-homo-estra-4,9,16-trien-3-one,
46) 17-Chloro-17aβ-hydroxy-13-ethyl-17a-homo-estra-4,9,11,16-tetraen-3-one,
47) 17-Chloro-17aβ-hydroxy-7α-methyl-13-ethyl-17a-homo-estra-4,9,11,16-tetraen-3-one
48) 4,17-Dichloro-17aβ-hydroxy-7α-methyl-13-ethyl-17a-homo-estra-4,9,11,16-tetraen-3-one,
49) 17-Chloro-17aβ-hydroxy-17a-homo-5α-androst-16-en-3-one (11),
50) 17-Chloro-17aβ-hydroxy-17a-homo-7α-methyl -5α-androst-16-en-3-one,
51) 17-Chloro-17aβ-hydroxy-17a-homo-2-hydroxymethylene-5α-androst-16-en-3-one (13),
52) 17-Chloro-17aβ-hydroxy-17a-homo-2α-methyl-5α-androst-16-en-3-one,
53) 17-Chloro-17aβ-hydroxy-17a-homo-1α-methyl-5α-androst-16-en-3-one,
54) 17-Chloro-17aβ-hydroxy-17a-homo-5α-androsta-2,16-diene,
55) 17-Chloro-17aβ-hydroxy-17a-homo-2-methyl-5α-androsta-2,16-diene,
56) 17-Chloro-17aβ-hydroxy-17a-homo-2-cyano-5α-androsta-2,16-diene,
57) 17-Chloro-17aβ-hydroxy-17a-homo-2-formyl-5α-androsta-2,16-diene,
58) 17-Chloro-17aβ-hydroxy-17a-homo-[2,3c]oxadiazole-5α-androst-16-ene,
59) 17-Chloro-17aβ-hydroxy-17a-homo-[3,2c]isoxazole-5α-androst-16-ene,
60) 17-Chloro-17aβ-hydroxy-17a-homo-[3,2c]pyrazole-5α-androst-16-ene,
61) 17-Chloro-17aβ-hydroxy-17a-homo-2β,3β-epithio-5α-androst-16-ene,
62) 17-Chloro-17aβ-hydroxy-17a-homo-2α,3α-epithio-5α-androst-16-ene,
63) 17-Chloro-17aβ-hydroxy-17a-homo-2-oxa-5α-androst-16-en-3-one (14),
64) 17-Chloro-17aβ-hydroxy-17a-homo-5α-androsta-1,16-dien-3-one (12),
65) 17-Chloro-17aβ-hydroxy-17a-homo-1-methyl-5α-androsta-1,16-dien-en-3-one,
66) 17-Chloro-17aβ-hydroxy-17a-homo-2-methyl-5α-androsta-1,16-dien-en-3-one,
67) 17-Chloro-17aα-methyl-17aβ-hydroxy-17a-homoandrosta-4,16-dien-3-one,
68) 17-Chloro-17aα-methyl-17aβ-hydroxy-7α-methyl-17a-homoandrosta-4,16-dien-3-one,
69) 17-Chloro-17aα-ethyl-17aβ-hydroxy-17a-homoandrosta-4,16-dien-3-one,
70) 17-Chloro-17aα-ethyl-17aβ-hydroxy-7α-methyl-17a-homoandrosta-4,16-dien-3-one,
71) 17-Chloro-17aα-ethinyl-17aβ-hydroxy-17a-homoandrosta-4,16-dien-3-one,
72) 17-Chloro-17aα-ethinyl-17aβ-hydroxy-7α-methyl-17a-homoandrosta-4,16-dien-3-one,
73) 17-Chloro-17aα-hydroxymethyl-17aβ-hydroxy-17a-homoandrosta-4,16-dien-3-one,
74) 17-Chloro-17aα-hydroxymethyl-17aβ-hydroxy-7α-methyl-17a-homoandrosta-4,16-dien-3-one,
75) 17-Chloro-17aα-chloromethyl-17aβ-hydroxy-17a-homoandrosta-4,16-dien-3-one,
76) 17-Chloro-17aα-chloromethyl-17aβ-hydroxy-7α-methyl-17a-homoandrosta-4,16-dien-3-one,
77) 17-Chloro-17aα-bromomethyl-17aβ-hydroxy-17a-homoandrosta-4,16-dien-3-one,
78) 17-Chloro-17aα-bromomethyl-17aβ-hydroxy-7α-methyl-17a-homoandrosta-4,16-dien-3-one,
79) 17-Chloro-17aα-cyanomethyl-17aβ-hydroxy-17a-homoandrosta-4,16-dien-3-one,
80) 17-Chloro-17aα-cyanomethyl-17aβ-hydroxy-7α-methyl-17a-homoandrosta-4,16-dien-3-one,
81) 17-Chloro-17aα-azidomethyl-17aβ-hydroxy-17a-homoandrosta-4,16-dien-3-one,
82) 17-Chloro-17aα-azidomethy-17aβ-hydroxy-7α-methyl-17a-homoandrosta-4,16-dien-3-one,
83) 17-Chloro-17aα-rhodanomethyl-17aβ-hydroxy-17a-homoandrosta-4,16-dien-3-one,
84) 17-Chloro-17aα-rhodanomethyl-17aβ-hydroxy-7α-methyl-17a-homoandrosta-4,16-dien-3-one,
85) 17-Chloro-17aα-methoxymethyl-17aβ-hydroxy-17a-homoandrosta-4,16-dien-3-one,
86) 17-Chloro-17aα-methoxymethyl-17aβ-hydroxy-7α-methyl-17a-homoandrosta-4,16-dien-3-one,
87) 17-Chloro-17aα-methyl-17aβ-hydroxy-17a-homo-estra-4,16-dien-3-one,
88) 17-Chloro-17aα-methyl-17aβ-hydroxy-7α-methyl-17a-homo-estra-4,16-dien-3-one,
89) 17-Chloro-17aα-ethyl-17aβ-hydroxy-17a-homo-estra-4,16-dien-3-one,
90) 17-Chloro-17aα-ethyl-17aβ-hydroxy-7α-methyl-17a-homo-estra-4,16-dien-3-one,
91) 17-Chloro-17aα-ethinyl-17aβ-hydroxy-17a-homo-estra-4,16-dien-3-one,
92) 17-Chloro-17aα-ethinyl-17aβ-hydroxy-7α-methyl-17a-homo -estra-4,16-dien -3-one,
93) 17-Chloro-17α-hydroxymethyl-17aβ-hydroxy -I17a-homo-estra-4,16-dien-3-one,
94) 17-Chloro-17α-hydroxymethyl-17aβ-hydroxy-7(-methyl-17a-homo -estra-4,16-dien-3-one,
95) 17-Chloro-17aα-chloromethyl-17aβ-hydroxy-17a-homo-estra-4,16-dien-3-one,
96) 17-Chloro-17aα-chloromethy-17aβ-hydroxy-7α-methyl-17a-homo-estra-4,16-dien-3-one,
97) 17-Chloro-17aα-bromomethyl-17aβ-hydroxy-17a-homo-estra-4,16-dien-3-one,
98) 17-Chloro-17aα-bromomethyl-17aβ-hydroxy-7α-methyl-17a-homo-estra-4,16-dien-3-one,
99) 17-Chloro-17aα-cyanomethyl-17aβ-hydroxy-17a-homo-estra-4,16-dien-3-one,
100) 17-Chloro-17aα-cyanomethyl-17aβ-hydroxy-7α-methyl-17a-homo-estra-4,16-dien-3-one,
101) 17-Chloro-17aα-azidomethyl-17aβ-hydroxy-17a-homo-estra4,16-dien-3-one,
102) 17-Chloro-17aα-azidomethyl-17aβ-hydroxy-7α-methyl-17a-homo-estra-4,16-dien-3-one,
103) 17-Chloro-17aα-rhodanomethyl-17aβ-hydroxy-17a-homo-estra-4,16-dien-3-one,
104) 17-Chloro-17aα-rhodanomethyl-17aβ-hydroxy-7α-methyl-17a-homo-estra-4,16-dien-3-one,
105) 17-Chloro-17aα-methoxymethyl-17aβ-hydroxy-17a-homo-estra-4,16-dien-3-one,
106) 17-Chloro-17aα-methoxymethyl-17aβ-hydroxy-7α-methyl-17a-homo-estra-4,16-dien-3-one.

22. Process for the production of compounds according to claim 1 with general formula I 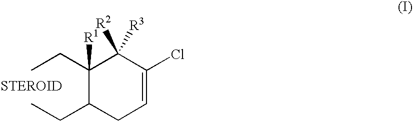
comprising
a) the reaction of the enol compounds of 17-ketones according to general formula II 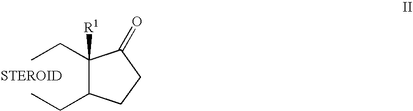
in which
R¹ and STEROID have the meaning that is given in claims 1 to 10, or can mean the following steroid bases for partial structures A-F
A: 6-Methoxy-3,5-cyclo-androstan-17-one,
B: 3,3-Dimethoxy-estr-5(10)-en-17-one,
18a-Homo-3,3-dimethoxy-estr-5(10)-en-17-one,
C,D,E,F: Epiandrosterone
with dichlorocarbene
to form compounds of general formula III 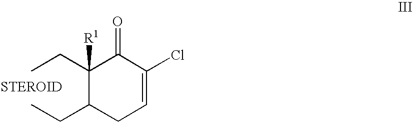
in which STEROID can mean the partial structures A-F or the above-mentioned steroid bases, and
b) reduction and optionally substitution of the compounds of formula III.

23. Process according to claim 22, whereby the functional groups that are contained in partial structures A-F or in the steroid bases are protected.

24. Process according to claim 22, wherein in Step b), the corresponding trialkylsilylenol ether derivatives of the compounds of formula II are used.

25. Pharmaceutical compositions that contain at least one compound according to claim 1.

26. Process for the production of pharmaceutical agents that contain compounds according to claim 1 for hormone replacement therapy (HRT) in men and women.

27. Process for the production of pharmaceutical agents that contain compounds according to claim 1 for birth control in men and women.

28. Process for the production of pharmaceutical agents that contain compounds according to claim 1 for treatment of hormone-dependent diseases in men and women.

29. Process according to claim 28, wherein the disease is endometriosis, breast cancer or hypogonadism.
